(19) United States
(12) Patent Application Publication
(10) Pub. No.: US 2011/0282298 A1
(43) Pub. Date: Nov. 17, 2011

(54) DEVICE FOR INJECTING FLUID ISOLATED FROM MICRONEEDLE HUB WITH DEAD-SPACE-REDUCING INSERT

(76) Inventors: Nadav Agian, Kfar Yona (IL); Yonni Shem-Tov, Holon (IL); Barak Panga, Moshav Ptahia (IL); Yoel Sefi, Dn Merom Hagalil (IL); Yotam Levin, Nes Ziona (IL)

(21) Appl. No.: 13/133,710

(22) PCT Filed: Dec. 9, 2009
(86) PCT No.: PCT/IB09/55635

§ 371 Date: Jul. 25, 2011

Related U.S. Application Data
(60) Provisional application No. 61/120,865, filed on Dec. 9, 2008.
(60) Provisional application No. 61/178,101, filed on May 14, 2009.

(51) Int. Cl. A61M 5/31; A61M 5/315; B32B 37/00; A61M 5/32

(52) U.S. Cl. 604/201; 604/240; 604/218; 156/60

(57) ABSTRACT

An injection system applicable to off-the-shelf syringes having a microneedle hub may reduce dead space and isolate the drug fluid from the microneedle chip to avoid compatibility issues. An insert may be disposed inside a male luer fitting and extend so that it is also disposed inside the female luer fitting using an insert. A septum may isolate the drug fluid from the microneedle chip in the hub prior to activation and the septum may, after activation, seal the triangular space in the hub to prevent leakage of fluid back to the syringe.

### PRIORITY INFORMATION

This patent application claims the benefit of U.S. Provisional Patent Application No. 61/120,865, filed Dec. 9, 2008. This patent application also claims the benefit of U.S. Provisional Patent Application No. 61/178,103 filed May 14, 2009.

### FIELD AND BACKGROUND OF THE INVENTION

The present invention relates to an injection device and, more particularly, to an injection device that reduces dead space in microneedle-syringe mating arrangements and/or that safely stores the fluid apart from the microneedle chip or other needle.

Applicants have previously developed an apparatus useful for shallow delivery of drug fluid through a flexible biological barrier such as the skin based on hollow and non-hollow microstructures, including microneedles, micropyramids and microprojections. For conciseness, all of these three above terms will be referred to herein generically as “microneedles” or “microneedle structures”. Furthermore, “needles” means at least one needle. Microneedles are generally referred to as miniature hollow needles in sizes usually less than approximately 1 mm. Microneedle material could be silicon (including various coats), metal, polymer and like materials known in micromachining and Micro Electro Mechanical Systems (“MEMS”). Typically, the microneedle structures are made from silicon single crystal by MEMS processes. Details of the structures and production techniques for these structures may be found in the following prior Nanopass patent publications which are hereby incorporated by reference in their entirety: (i) U.S. Pat. No. 6,533,949 issued Mar. 18, 2003; (ii) US Patent Publication no. 20090069788 published Mar. 14, 2009 for patent application no. 11/719,541 (PCT Patent Publication No. WO 2006/054280); and (iii) U.S. patent publication no. 20080091226 published Apr. 17, 2008 for patent application Ser. No. 11/549,982. Micro-needles may be used for intradermal (ID) injections of fluids and such injections may facilitate dose sparing. For example it has been previously demonstrated that reduced doses of a vaccine delivered intradermally can produce equivalent immune responses (or immunogenicity) with the full dose (and volume) of intra-muscular (IM) injection. See Van Damme, P., et al. Safety and Efficacy of a Novel Microneedle Device for Dose Sparing Intradermal Influenza Vaccination in Healthy Adults, Vaccine (2008), doi:10.1016/j.vaccine.2008.10.077.

Microneedles are often used with prefilled syringes. The pre-filled syringes are usually made of glass tube containers. Specific structures designed on top of such glass tubes (including holders, fixed needles and shields, and injection mechanisms and devices) are not easily attached or bonded to these glass syringes because of the nature of the materials.

Drug fluids stored in pre-filled syringes may or may not be incompatible to some extent with silicon microneedle structures but in either case the compatiblity issue is sufficiently strong to trigger a need for additional testing. Accordingly, if the microneedle and the drug are in contact, or at risk of being in contact, safety and effecacy guidelines may require testing the drug for long term stability. This can be inconvenient and/or expensive and would require re-approval of a particular reservoir of drug fluid.

Therefore, there is a need to create a barrier between the drug fluid and the microneedle chip in a pre-filled syringe such that compatibility issues do not arise, and so that there is no need for long term stability testing and re-approval of a reservoir due to the combination with a microneedle delivery configuration.

In addition to the compatibility issue, another issue arises regarding injection devices, particularly injection devices having microneedles. Injection devices usually retain some volume of medication in spaces within the device before and following an injection. These spaces are being referred to as the “dead space” of the injection device. For example, a typical dead space of an interchangeable needle-syringe assembly with leer fittings may be approximately 60-100 micro-liters (and typically 80 micro-liters)

Prior art injection systems include U.S. Pat. No. 5,902,271 to Jentzen discloses devices for achieving interchangeable needles while maintaining low residual medication. In one embodiment, the plunger sealing elastomer is designed with an elongated nose tip to expel residual medication from the syringe nozzle. U.S. Patent Publication no. US 2008/0033347 to D'Arrigo et al discloses a non luer syringe and detachable needle assembly having reduced dead space. U.S. Published Patent Application Pub. No. US 2003/0181863 to Ackley et al discloses an adapter for the transport of fluids with a microneedle device. The adapter can include a seal through which a syringe needle is inserted to deliver fluid from the syringe into a fluid cavity in the adapter.

In cases of dose sparing intradermal injections, 80 micro-liters of dead space may almost double the required medication (e.g., ˜180 μl is required for practically injecting 100 μl). Thus, there is an even greater need to reduce dead space in such cases, particularly in microneedle systems for intradermal injections.

### SUMMARY OF THE PRESENT INVENTION

One aspect of the present invention is directed to a coupler that reduces dead space for fluids in an injection device, comprising a male fitting of an injection container; a female fitting having a proximal end for fitting inside the male fitting; and an insert disposed within the proximal end of the female fitting and disposed within the male fitting for minimizing fluid remaining in the injection container and in the female fitting after injection, the insert shaped so as to maintain a flow path between the injection container and a needle attached to the female fitting.

In a further aspect of the present invention, there is provided a coupler in an injection device, comprising a male luer-lock fitting of a syringe, a female luer-lock fitting having a proximal end for fitting inside the male luer-lock fitting; and an insert disposed within and attached to the proximal end of the female luer-lock fitting and disposed within the male luer-lock fitting for minimizing fluid remaining in the syringe and in the female luer-lock fitting after injection, the insert shaped so as to maintain a flow path between the syringe and a needle attached to the female fitting.

In a still further aspect of the present invention, there is provided a pre-filled injection device, comprising a microneedle hub; a syringe pre-filled with a fluid and having a needle at a distal end of the syringe; an activation connector connected at a first end of the activation connector to a proximal end of the microneedle hub and connected to the distal end of the syringe at a second end of the activation connector; a septum fitted in the microneedle hub such that in a stored position of the injection device the needle extends into the microneedle hub without piercing the entire septum and in an active position of the injection device the needle penetrates the septum entirely to maintain a flow channel between the fluid and a microneedle chip in the microneedle hub.

A still further aspect of the present invention is directed to a pre-filled injection device, comprising an injection container pre-filled with a fluid, an insert having a diaphragm associated therewith, a proximal end of the insert fitted to a distal end of the injection container, the diaphragm sealing the distal end of the injection container; a needle unit mated to a distal end of the insert so that activation of the needle unit moves a projecting element of the needle unit axially from a stored position in which the projecting element does not touch the diaphragm to an active position in which the projecting element pierces the diaphragm.

In a further aspect of the present invention, there is presented a microneedle injection device, comprising a disposable syringe filled with a fluid and having an aspiration needle hub at a distal end of the syringe, the aspiration needle hub having an aspiration needle projecting out of the aspiration needle hub; a microneedle hub including a septum disposed in the microneedle hub, the microneedle hub shaped to mate with the aspiration needle hub, the septum sealing a flow path between the microneedle hub and the syringe to prevent backflow of fluid into the syringe.

A further aspect of the present invention involves a method of preparing a microneedle injection device, comprising using an aspiration needle affixed to a distal end of a disposable syringe to fill the syringe with a drug, the aspiration needle projecting from an aspiration needle hub; placing a microneedle hub having a septum disposed therein onto the aspiration needle hub so that the aspiration needle pierces the septum, the septum sealing a flow path between the microneedle hub and the syringe.

In a still further aspect of the present invention, there is presented a coupler for an injection device, comprising a male Luer-lock fitting of a syringe, the syringe not having a needle directly attached thereto; a female leer-lock fitting having a proximal end for fitting inside the male luer-lock fitting, the female leer-lock fitting also having a pipe integrally formed thereto, the pipe disposed within and extending out of a proximal end of the female luer-lock fitting, the pipe shaped to attach to the male luer-lock fitting so as to form a sealed flow path from the syringe through the male luer-lock fitting to the female luer-lock fitting that bypasses dead space in the male luer-lock fitting and in the female luer-lock fitting.

In one further aspect of the present invention, a female bier-lock fitting for a coupler of an injection device comprises a male luer-lock fitting of a syringe, the syringe not having a needle directly attached thereto; a female luer-lock fitting having a pipe integrally formed, thereto, the pipe disposed within and extending out of a proximal end of the female luer-lock fitting, the pipe shaped to attach to the male luer-lock fitting so as to form a sealed flow path from the syringe through the male luer-lock fitting to the female luer-lock fitting that bypasses dead space in the male luer-lock fitting and in the female luer-lock fitting.

In a still further aspect of the present invention, there is presented an injection device, comprising an injection container including a barrel; a first plunger at an end of a piston and sealing the barrel from fluid flow; a second plunger downstream of the first plunger and sealing the barrel from fluid flow; and fluid separating the first plunger and the second plunger prior to injection, the barrel, downstream of the second plunger, having a bypass channel so that the second plunger no longer seals against fluid flow when the first plunger is depressed.

A further aspect of the present invention involves a pre-filled injection device, comprising an injection container having a barrel pre-filled with a fluid downstream of a plunger sealing a top boundary of the fluid in a chamber, the injection container also having a diaphragm at a distal end of the barrel sealing the fluid at a lower boundary; a microneedle hub attached to a distal end of the injection container; the diaphragm breakable from pressure in the chamber when the piston is depressed for injection.

In a still further aspect of the present invention, there is provided an injection device, comprising a pre-filled syringe including a glass barrel; a microneedle hub containing a silicon microneedle chip, the silicon microneedle chip bonded directly to the glass barrel using a bonding process selected from the group consisting of diffusion and adhesive bonding.

A further aspect of the present invention involves a method used in making an injection device, comprising providing a syringe having a glass barrel; bonding the glass barrel to a silicon microneedle chip of a microneedle hub using a process selected from the group consisting of anodic bonding, thermal bonding and diffusion

These and other features, aspects and advantages of the present invention will become better understood with reference to the following drawings, descriptions and claims.

### BRIEF DESCRIPTION OF THE DRAWINGS

Various embodiments are described herein by way of example only, with reference to the accompanying drawings, wherein:

FIG. 1A is a perspective view of a portion of a coupler including its insert disposed inside a female fitting, with the male fitting omitted, in accordance with one embodiment of the present invention;

FIG. 1B is a sectional view of a coupler in accordance with one embodiment of the present invention;

FIG. 1C is a sectional view as in FIG. 1B with the “dead space” darkened, in accordance with one embodiment of the present invention;

FIG. 1D is a perspective view of a wide-head insert and a female fitting prior to assembly of the two together, in accordance with one embodiment of the present invention;

FIG. 1F is a perspective view of a wide-head insert already assembled inside a female fitting in accordance with one embodiment of the present invention;

FIG. 1F is a side view of the insert and female fitting of FIG. 1E;

FIG. 2 is an exploded view of an injection device, in accordance with one embodiment of the present invention;

FIG. 3A is a sectional view of an injection device having one form of an activation connector prior to activation, in accordance with one embodiment of the present invention;

FIG. 3B is an enlarged view of the front of the device shown in FIG. 3A;

FIG. 3C is a sectional view of the injection device shown in FIG. 3A but after activation;

FIG. 3D is an enlarged view of the front of the device shown in FIG. 3C;

FIG. 4 is a partial sectional view of n injection system having a rotational mechanism for the activation connector shown before and after activation;

FIG. 5A is an injection device in accordance with one embodiment of the present invention;

FIG. 5B is an exploded view of the device shown in FIG. 5A;

FIG. 5C is an enlarged partial sectional view of the area circled in FIG. 5A showing the microneedle hub, transparent microneedle cover and part of the syringe;

FIG. 6A is a rear perspective view of a microneedle hub which is a female luer fitting used in an injection device in accordance with one embodiment of the present invention;

FIG. 6B is a rear perspective view of the female luer fitting of FIG. 6A;

FIG. 6C is a partial sectional view showing the mating of the female luer fitting with the male fitting in accordance with the embodiment of FIG. 6A and FIG. 6B;

FIG. 7A is an exploded view of an injection system in accordance with one embodiment of the present invention;

FIG. 7B shows sectional and perspective views of an injection device in accordance with one embodiment of the present invention before and after activation;

FIG. 7C is an enlarged fragmentary view of the top left portion of FIG. 7B showing the device in locked/stored position prior to activation and in section;

FIG. 7D is an enlarged fragmentary view of the top right portion of FIG. 7B showing the device after activation and in section;

FIG. 8A is a front view and an enlarged fragmentary view of an injection device in accordance with one embodiment of the present invention prior to injection;

FIG. 8B is a front view and an enlarged fragmentary view of an injection device in accordance with one embodiment of the present invention during injection;

FIG. 8C1 and 8C2 are top views of the cross-section of the barrel of the syringe showing an alternative embodiment of the bypass channel used in the device of FIG. 8A wherein FIG. 8C1 is the cross-section throughout most of the barrel and 8C2 is the cross-section at the bypass channel;

FIG. 9A is a sectional view of an injection device in accordance with one embodiment of the present invention prior to injection;

FIG. 9B is a sectional view of an injection device of FIG. 9A during injection;

FIG. 10A is a side view of a syringe having a microneedle chip bonded directly to the glass barrel of the syringe; and

FIG. 10B is an enlarged fragmentary view of the portion of FIG. 10A in which the mating between the glass barrel and the silicon chip occurs.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention generally provides an injection system for off-the-shelf syringes and may also have a microneedle hub having a microneedle chip. A coupler that may include an insert may be disposed in part inside a male luer lock fitting and in part inside a female luer lock fitting. The coupler may reduce dead space in both the male and female fittings. In addition, a septum may isolate the drug fluid from the microneedle chip in the hub prior to activation and the septum may, after activation, seal the triangular space in the hub to prevent leakage of fluid back to the syringe.

In contrast to the prior art, in which the residual volume of drug fluid after injection may approach 50% of the original volume of drug fluid inside the syringe (i.e. 80 microliters wasted for 100 microliters delivered), especially with low dose injection systems that utilize microneedles, the injection device of the present invention fitted with microneedles may have a residual volume of only 10 or fewer microliters associated with 100 microliters of drug fluid that is injected. This means that only 110 microliters would be needed to inject 100 microliters rather than 180 microliters, as found in the prior art. In further contrast to the prior art, in which drug fluid may be at risk of being in contact with a silicon microneedle chip, for example where there is no septum or there is a septum that only prevents backflow of fluid into the syringe after activation, the injection system of the present invention may isolate the drug fluid from the microneedle chip prior to injection in one of several ways—using a bi-functional septum, a pierceable membrane or a double plunger. In further contrast to the prior art, in which injection devices that are designed to reduce dead space accomplish this by using uniquely designed syringes or uniquely designed plungers that cannot be adapted to off-the-shelf commercially available injection devices, the injection device of the present invention contains novel elements (i.e. insert, septum, activation connector etc.) that are designed to be adapted to off-the-shelf commercially available standard syringes. In further contrast to the prior art pre-filled syringes, in which it is necessary to achieve the very difficult task of blocking/covering a delicate microneedle (and all its individual microneedles) with a rubber shield to prevent leakage during shelf life, which is very difficult to do without breakage, and to enable filling, the injection device of the present invention avoids having to do this because the fluid is isolated from the microneedles as a result of the rubber septum functioning as a sealing member prior to activation by sealing the needle of the syringe. In further contrast to the prior art, in which production of the glass syringe barrel involves glass forming by rotation, which yields a round, symmetric syringe tip, which shape is difficult to integrate with a square or non-symmetrically shaped microneedle array and necessitates tight dimension tolerances, the injection device of the present invention, by using a plastic microneedle hub connected to a regular glass syringe, may reduce these production and product design limitations.

In still further contrast to the prior art, in which a regular aspiration needle has to be disconnected from a syringe before placing a microneedle hub onto the syringe, a step which uses up precious time and effort, in one embodiment of the injection device of the present invention a microneedle injection device may include a microneedle hub shaped to fit directly over an aspiration needle hub having an aspiration needle thus transforming a standard hypodermic needle fixed directly on a syringe or connected to its hub into an ID microneedle without the need to disconnect the aspiration needle:

In contrast to the prior art, in which the befits of shallow delivery are not present or in which these benefits are limited, the injection devices of the present invention may further improve ease of use for the health care provider since there is no need to withdraw the fluid from a vial, no need to use and exchange needles, which improves safety and sharply reduces exposure, faster procedure time (which is especially important for heavy load clinics or mass vaccination); easier use for self administration; and more accurate dosing.

In contrast to the prior art, the injection device of the present invention and methods using it provide biological benefits to the patient including enhanced vaccination, dose sparing, reduction of boost requirements, improved kinetics; usability benefits—lesser pain and needle phobia, a more reliable technique for consistent shallow delivery depth than hypodermics or other systems, and user and patient safety: no ability to penetrate or harm deeper tissue, no ability to produce an IV injection); more reliable skin diagnostics.

It should be understood that the term “drug fluid” shall be understood in this patent application to include fluid compounds that may be injected, for example injected into a person or animal, even if for whatever reason such fluid would not normally be considered a drug.

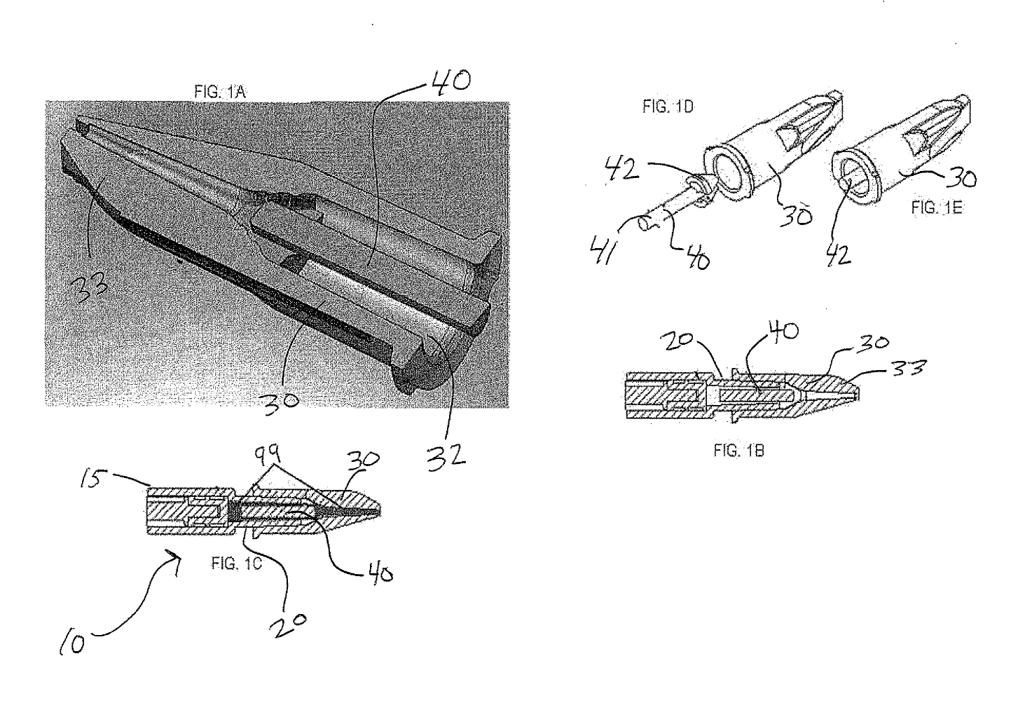

As seen in FIG. 1A, 1B, 1C, 1D and 1E, a coupler 10 that reduces dead space for fluids in an injection device is provided. Coupler 10 may include a male fitting 20 of a syringe 15, the male fitting 20 designed and shaped for receipt of a female fitting. For example, male fitting 20 may be a male luer-lock fitting of a syringe 15. Male fitting 20 may be a nozzle of the syringe 15. Coupler 10 may also include a female fitting 30 whose proximal end 32 may fit inside male fitting 20. Female fitting 30 may be a female luer lock fitting of the syringe. Female fitting 30 may be viewed as a microneedle hub 30 and may have a distal end 33 attached to a microneedle chip (not shown).

Coupler 10 may include a dose-sparing insert 40 disposed within the proximal end of female fitting 30 and also disposed within male fitting 20. Insert 40 may serve to minimize fluid remaining in the syringe and in the female fitting after injection.

Dose-sparing insert 40 may be emplaced within an interchangeable luer mating arrangement of a microneedle device. Insert 40 may be a block of material or a combination of materials that fills dead spaces 99 in the mating arrangement, which may be a luer mating arrangement. The insert may reduce dead spaces both at the hub (female luer) and at the syringe nozzle (male luer).

As shown in the sectional view of FIG. 1B, insert 40 is typically designed with a wide distal end (herein “head”) 41 and a narrow proximal end (herein “tail”) 42. The head 41 of insert 40 typically is disposed within and fills spaces inside the female hub 30 and the tail 42 of insert 40 typically is disposed within and fills spaces inside a syringe male tip. In one embodiment, a greater portion of the length of insert 40 may be disposed inside male fitting 20 that is disposed inside female fitting 30. As shown in FIG. 1A, for example, more than three-quarters of the volume of insert 40, and of the length of insert 40, may be disposed inside the male fitting 20.

Insert 40 may be attached to a proximal end of the female luer-lock fitting 30 via a friction fit or may be integrally joined thereto. In the latter case, insert 40 and, the microneedle hub 30 may be designed as one piece of molded material (e.g., it could either be attached, approximated or integrally formed), or can be designed as one piece of several materials molded for example by co-injection or over-molding techniques. In certain other embodiments, head 41 of insert 40 may be designed to fit the female hub 30 such that the mating between them could be facilitated by pressure only (for example friction fit); or a rubber insert may be designed to be attached to the female microneedle interface/hub 30 using adhesive, in which case insert 40 and hub 30 may also be shaped to include bonding points, i.e. places such as grooves or channels for the adhesive to be applied during production.

Insert 40 may be shaped to maintain a flow path between the syringe and a needle attached to female fitting, such as microneedle. One way to accomplish this is to have insert 40 designed with exterior flow channels or grooves to maintain open flow paths between the syringe and the microneedles. The flow path is typically at the exterior of the insert.

Insert 40 may be designed to fit to a variety of different micro needle hubs—for example micro needle hubs with luer fittings as well as micro needle hubs that have other shapes. Insert 40 may also be designed to fit different syringe types that include luer slip or luer lock connectors.

Insert 40 may be made of soft materials such as rubber or silicone or plastic (or other polymeric) materials so that insert 40 can contract upon contact with the male nozzle in cases where the male nozzle reaches full friction fit only in a deeper position within the female luer. Typically, insert 40 may be made of biocompatible material. The insert 40 may also be coated, as required by the medicine (i.e. the drug fluid) or by other considerations, for example to ensure smooth flow, improve compatibility, shelf life and similar considerations or to reduce leachables and extractables, to improve (or reduce, as the case may be) fluid flow intentionally.

Insert 40 may be placed within the female luer during assembly of the injection device. Alternatively, the injection device may be supplied to customers with insert 40 already inside. In a further alternative, inserts 40 may be supplied to customers separated from and along with injection devices, and the customer may insert the insert into the injection device prior to use.

The coupler 10 and insert 40 of the present invention is not limited to injection devices having microneedles. For example, the insert 40 may be used with other fittings and other devices. For example, the insert 40 can be used with hypodermic needles connected to a syringe via luer connection or other less standard connections.

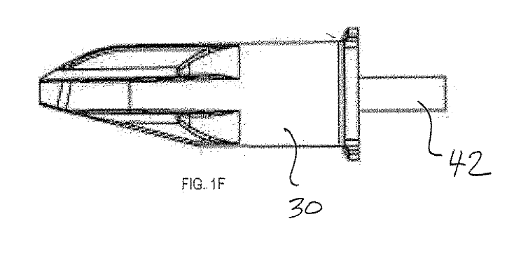

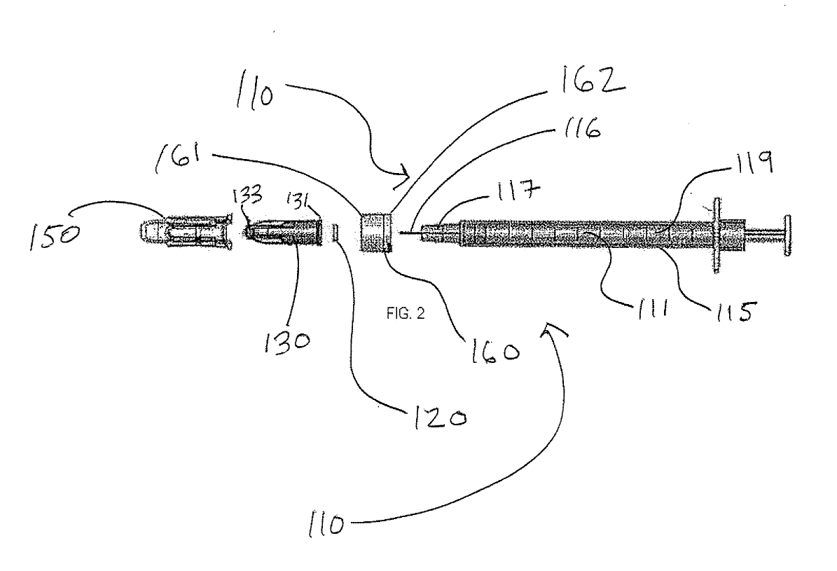

In another possible configuration of the present invention, as seen in FIG. 2, the injection device of the present invention may be designed to fit a standard pre-filled injection device. FIG. 2 is an exploded view of the injection device 110 including a pre-filled syringe Pre-filled injection device 110 may include a microneedle hub 130, a syringe 115 pre-filled with a fluid, the syringe 115 having a needle 116 at a distal end 117 of the syringe 115. The microneedle hub 130 may include a microneedle chip 133 that includes one or more microneedles (not visible) fitted therein. In any embodiment, microneedle hub 130 may be fitted with a microneedle cover 150 to protect it.

Injection device 110 may differ from a standard pre-filled injection device, among other ways, in that it may also have an activation connector 160. A proximal end 131 of microneedle hub 130 may be disposed within or connected to a first end 161 of the activation connector 160. Distal end 117 of syringe 115 may be disposed within or connected to a second end 162 of activation connector 160. Accordingly, activation connector 160 may enable connection to syringe 115 from one side.

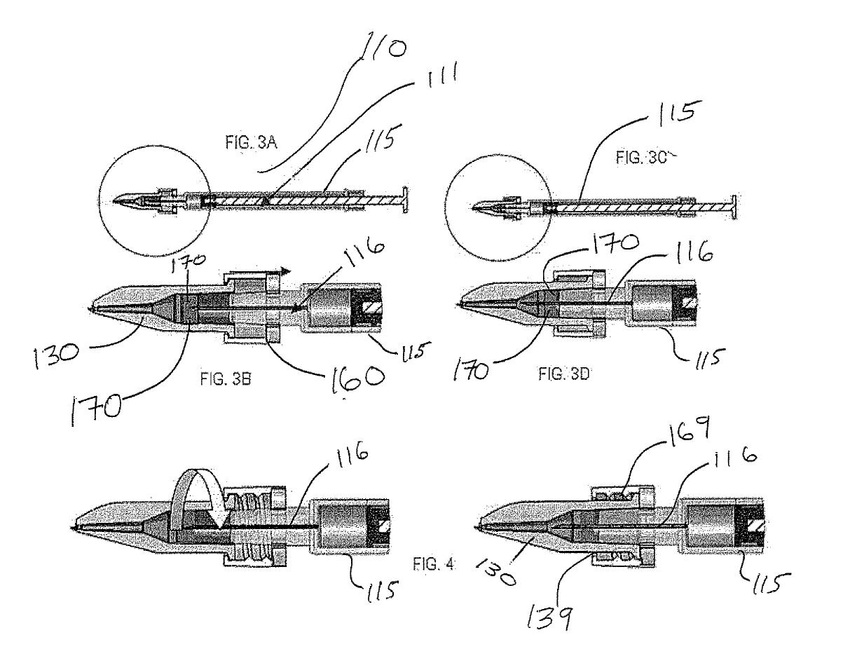

Injection device 110 may also include a septum 170 or other penetrable sealant 170 fitted in microneedle hub 130 in such a way that in a stored position of the injection device 110, as shown for example in FIG. 3B, needle 116 may extend into microneedle hub 130 and partially penetrate septum 170 without entirely piercing through to the other end of septum 170. In an active position of injection device 110 (i.e. after or during injection) as shown for example in FIG. 3D, needle 116 may extend through and pierce through the full thickness or length of septum 170 so as to maintain a flow channel between the fluid 111 in the barrel of syringe 115 and microneedle chip 133 in microneedle hub 130.

Accordingly, septum 170 may be said to be hi-functional. Before activation, septum 170 may serve the function of isolating drug fluid 111 pre-filled in the barrel 119 of syringe 115 from microneedle chip 133, with which it may have compatibility issues and to generally preserve the fluid 111 to avoid stability and leakage issues. Septum 170 may also act as a seal for needle 116 of syringe 115, enabling filling of syringe 115 without leakage. After activation, septum 170 may serve its second function, namely to seal the generally triangular space of microneedle hub 130 to prevent drug fluid 111 from leaking backward into syringe 115.

Septum 170 may be assembled in microneedle hub 130. Before activation, septum 170 may be used as a seal for the syringe needle 116, enabling filling and preventing drug leakage.

FIGS. 3A-3D show one embodiment of effectuating movement of activation connector 160 relative to syringe 115. In this case, the proximal end of microneedle hub 130 may be fitted into activation connector 160 by a frictional fit that may allow axial adjustment of microneedle hub 130 relative to syringe 115. The user may then press back microneedle hub 130 towards syringe 115 in order to pierce the septum 170 and enable drug flow through to the microneedles. FIGS. 3A-3B depict the injection device prior to activation and FIGS. 3C-3D show the device after activation

Movement of activation connector 160 relative to syringe 115 may also be accomplished by other means. For example, in one method of operation shown in FIG. 4A and FIG. 4B, in order to effectuate movement of activation connector 160 relative to syringe 115, activation connector 160 and microneedle hub 130 may have mating threads 169, 139 to allow rotational movement of microneedle hub 130 relative to activation connector 160 and thereby linearly move microneedle hub 130 relative to syringe 115.

In all embodiments, the injection device (i.e. 10 or 110 or 120 etc.) of the present invention may have a low residual volume of drug fluid. For example, the device of the present invention may have less than 10 microliters of fluid remaining after injection.

Any of the injection devices of the present invention may also be adapted to fit with a standard pre-filled syringe having stacked needles (i.e. glass or plastic).

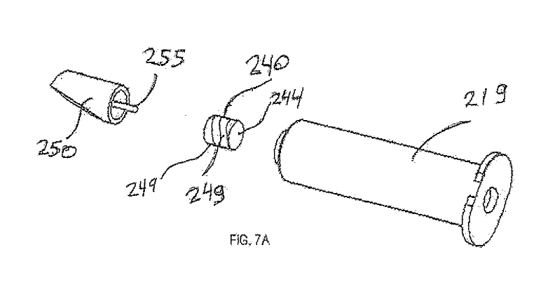

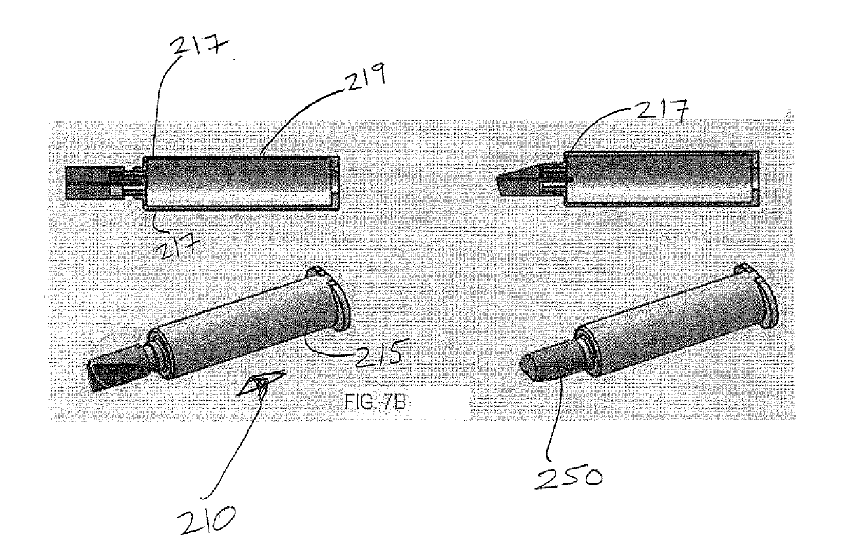

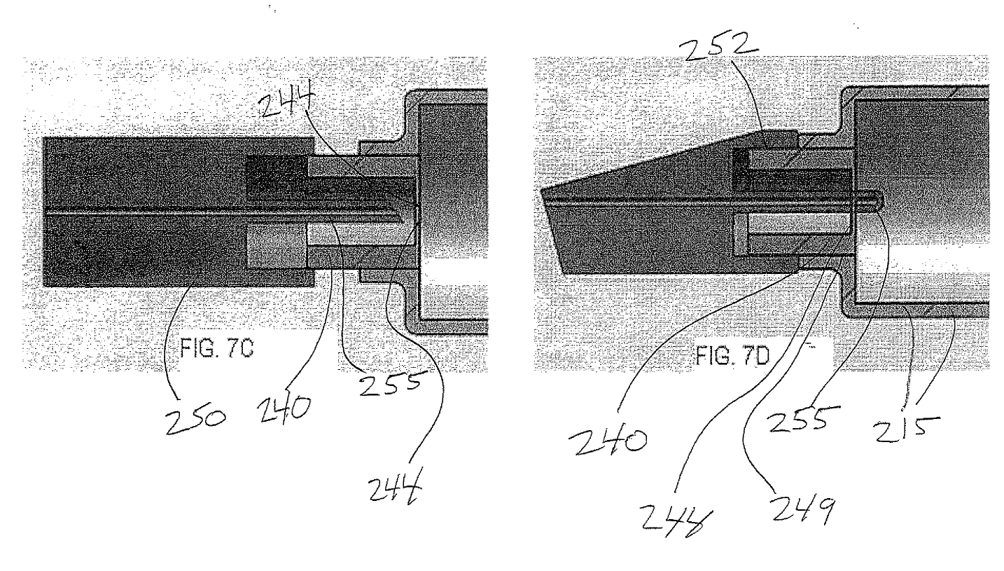

In another configuration of the present invention shown in FIG. 7A-7D, the injection device may be a pre-filled injection device 210, comprising a syringe 215 whose barrel 219, which may be made out of glass, may be pre-filled with a fluid 211. Not shown are the cover of the microneedle unit, or the plunger and rod of the syringe.

As seen in FIG. 7A, device 210 may include an insert 240, which may be made of plastic. Insert 240 may have a diaphragm 244, which may be made of rubber, associated therewith. Insert 240 may have an external thread 249. A proximal end 241 of insert 240 may be fitted to a distal end 217 of syringe 215. Diaphragm 244 may seal distal end 217 of syringe 215. In some embodiments, plastic insert 240 may be integrated to the glass barrel 219 in production phase, using adhesive or other kind of bonding.

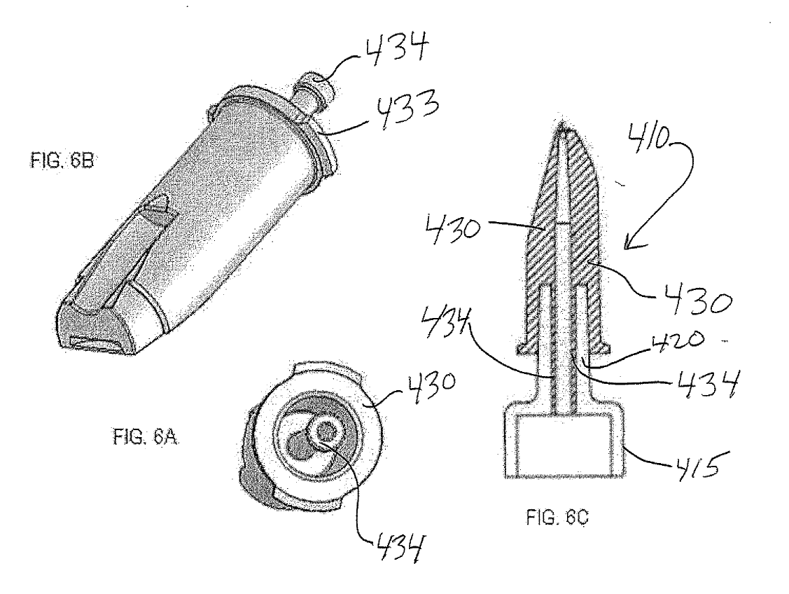

As shown in FIGS. 7A, 7B, 7C and 7D, device 210 may include a microneedle hub or unit 250 that may have a projecting punching element 255 at its proximal end. Projecting punching element may be hollow and sharp enough to pierce a membrane, as described. Microneedle unit 250 may have a microneedle chip (not shown) at a distal end of microneedle unit 250. Microneedle unit 250 may be mated to a distal end 241 of insert 240 so that activation of the microneedle unit 250 may move hollow projecting element 255 (i.e. plastic or metal) of needle unit 250 axially from a stored position (see FIG. 7C and left portion of FIG. 7B) in which projecting element 255 does not reach or touch diaphragm 244 to an active position in which projecting element 255 pierces diaphragm 244 (see FIG. 7D and right portion of FIG. 7B). Microneedle unit 250 may have an internal threading 252 (see FIG. 6D) and may be mated to distal end 241 of insert 240 by external threading 249 of insert 240 as shown in FIG. 7A. In that case, activation of microneedle unit 250 may be accomplished by a user's rotation of needle unit 250 such that it is screwed to the external threading 249 of insert 240. This action accomplishes a linear movement of the punching element 255 toward diaphragm 244, punches through it and enables drug flow through inside of hollow projecting element 255 of microneedle unit 250 and into the microneedles chip (not shown).

In certain embodiments, activation of microneedle unit 250 may occur by means other than the screwing mechanism described. For example, activation may occur by pushing the microneedle unit 250 backwards towards syringe 215 or sliding or other mechanisms known in the art.

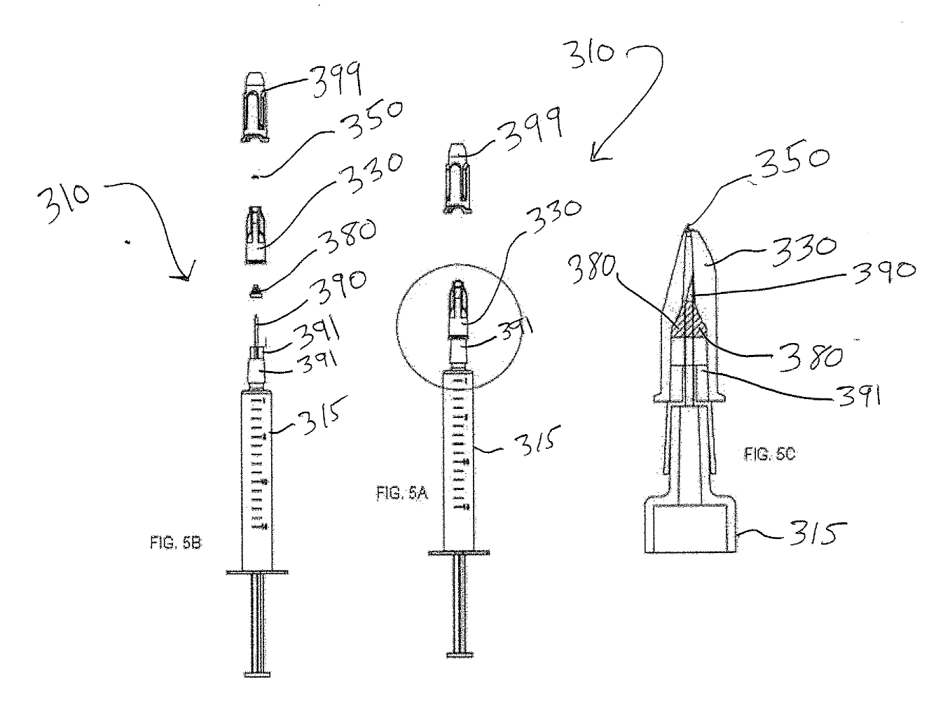

In a still further configuration of the present invention, there is shown in FIG. 5A, 5B and FIG. 5C, a microneedle injection device 310 that dispenses with the previously required step of removing an aspiration needle before placing a microneedle chip on to a syringe, for example a disposable syringe. This step normally occurs after an aspiration needle is affixed to the syringe and then inserted into a vial to draw fluid into a syringe (by pulling back the plunger). Normally, the health care worker must first remove the aspiration needle and only then place the microneedle hub onto the syringe. With device 310 there is no need to remove the aspiration needle prior to placing the microneedle hub onto the syringe because device 310 of the present invention transforms the standard hypodermic needle fixed directly on a syringe or connected to a hub (i.e. and aspiration needle hub) into an ID microneedle.

Device 310 may include a microneedle hub 330 and a disposable syringe 315 that may be filled with a fluid 311. Syringe 315 may have an aspiration needle hub 391 having aspiration needle 390 projecting out from aspiration needle hub 391 at a distal end of syringe 315. Aspiration needle hub 391 may be plastic. Microneedle hub 330 may have disposed within it a septum 380 which may be located at a proximal end 333 of microneedle hub 330. As shown in FIG. 5C, aspiration needle hub 391 may fit directly into microneedle hub 330. Septum 380 may seal a flow path between microneedle hub 330 and syringe 315. As seen in FIG. 5A and FIG. 5B, hub 330 may also have a cover 399. As seen in FIG. 5B, microneedle hub may include a microneedle chip 350 at a distal end of microneedle hub 330.

An example of a possible method of operation using this device 310 may be the following steps: (a) Drawing the drug using a standard needle; (b) Opening the microneedle device package; (c) Connecting the aspiration needle into the JO microneedle device, without taking it out from its pack, to reduce the risk of needle stick; (d) Removing the microneedle device shield; and (e) Performing ID injection

In a still further configuration, as shown in FIG. 6A, FIG. 6B and FIG. 6C, the present invention may be viewed as a coupler 410 for an injection device involving a syringe that does not have a needle directly attached thereto. As seen in FIG. 6C, coupler 410 may include a male fitting, such as a male luer-lock fitting 420 of a syringe. As seen in FIG. 6B, female luer-lock fitting 430 may have a proximal end 433 for fitting inside male luer-lock fitting 420.

As seen in FIG. 6A, 6B, 6C, female luer-lock fitting 430 may also have a pipe 434. Pipe 434 may be integrally formed to the inside of female luer-lock fitting 430 or may be attached thereto including as a separate insert. Pipe 434 may be disposed within the proximal end 433 of female luer-lock fitting 430 and, as seen in FIG. 6B and FIG. 6C, may extend out of proximal end 433 of female fitting 430. As shown in FIG. 6C, pipe 434 may attach and fit into male luer-lock fitting 420.

Coupler 410 may utilize pipe 434 so that a continuous sealed flow path of fluid is formed from the syringe to the male fitting 420 and through to female luer-lock fitting 430. The flow path may bypass exterior dead space (i.e. space outside pipe 434) in male luer-lock fitting 420 and in the cavity of female luer-lock fitting 430. The dead spaces around the pipe 434 may form closed air pockets expelling (at least some, if not the majority or all) the fluids during injection.

A typical dead space using device 410 with pipe 434 in accordance with one embodiment of the present invention for a luer arrangement may be ˜15 microliters compared to ˜80 microliters in standard luer arrangements. The pipe 434 or bypass channel can include additional sealing materials to improve sealing and prevent flow of the fluid into the cavity around the by pass channel. The seal can be designed at the opening of the bypass channel, anywhere around it, or even on the male unit of the syringe.

The present invention may also be viewed as a novel female luer-lock fitting 430 by itself, as seen in FIG. 6A and FIG. 6B, for a coupler of an injection device. As such, female luer-lock fitting 430 may have a proximal end 433 for fitting inside a male luer-lock fitting of a syringe that does not have a needle directly attached to it. Female luer-lock fitting 430 may also have a pipe 434 integrally formed thereto or attached thereto. As explained, pipe 434 may be disposed within and extend out of proximal end 433 of the female luer-lock fitting 430 and pipe 434 may be shaped to attach to the male luer-lock fitting so as to form a sealed flow path from the syringe through the male luer-lock fitting to the female luer-lock fitting 430 that bypasses dead space in the male luer-lock fitting and in the female luer-lock fitting 430.

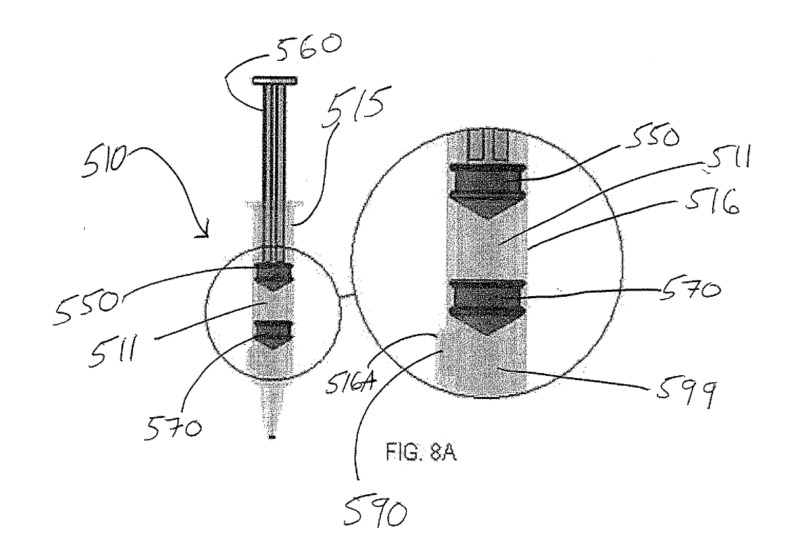

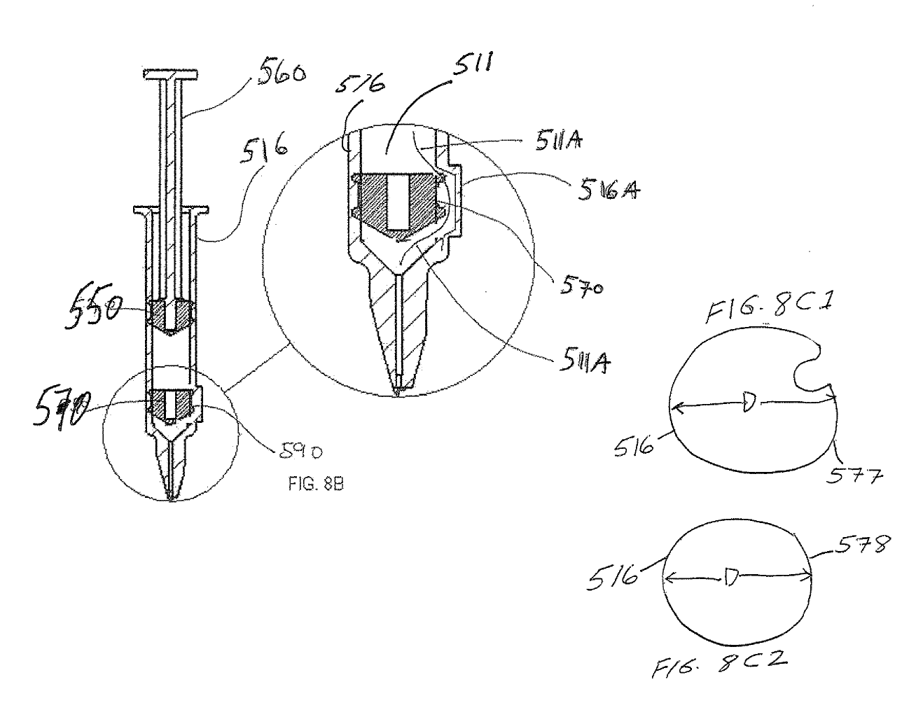

Other configurations of the present invention may be used to isolate the drug fluid from a microneedle chip or other needle. For example, in one configuration of the injection device of the present invention shown in FIG. 8A and FIG. 8B, an injection device 510 shown in FIG. 8A prior to injection comprises a syringe 515 including a barrel 516 and includes a first plunger 550 at an end of a piston 560, the first plunger 550 or gasket sealing barrel 516 from fluid flow. A second plunger 570 or gasket may be located downstream of the first plunger 550. The term “downstream” refers to the direction from the top of the syringe 515 to the bottom of the syringe 515. Second plunger 570 may seal barrel 516 from fluid flow. Prior to injection, as seen in FIG. 8A, fluid 511 may be present between first plunger 550 and second plunger 570 and both first plunger 550 and second plunger 570 seal and trap this fluid 511 within the space in barrel 516 between the two plungers 550, 570. As seen in FIG. 8B, during injection, fluid 511 travels through bypass channel 590 which in this case is a wider diameter portion 516A of barrel 516 of syringe 515.

Syringe 515 may be pre-filled with the fluid and may have attached to its distal end a microneedle hub. Between second plunger 570 and the needle there may be a minimal volume of air 599. When applying force on the syringe rod during injection, the developed pressure may push second plunger 570 into a wider inner diameter of barrel 516 or to a region provided with a bypass channel, thereby bypassing the seal, and enabling drug flow through the needle. The injection phase may therefore be the first time the drug is in contact with the needle/s.

Accordingly, downstream of second plunger 570, barrel 516 may have a bypass channel 590. As noted, the term “bypass channel 590” may include structures in which barrel 516 is simply wider at some point downstream of second plunger 570, the result being that second plunger 570 no longer seals against fluid flow after the first plunger 550 is depressed enough to cause second plunger 570 to be situated within the wider portion 516A of barrel 516.

Further, the term “bypass channel 590” may also include structures in which barrel 516 is shaped differently further downstream than its initial starting position shown in FIG. 8A. In this case also this causes second plunger 570 to no longer seal against fluid flow after the first plunger 550 is depressed enough to cause second plunger 570 to be situated in the differently shaped portion of barrel 516. In an alternative embodiment, one particular example of a change in shape of barrel 516 may be a case in which (see FIG. 8C1 and FIG. a female luer-lock fitting for a coupler of an injection device comprises a male luer-lock fitting of a syringe, the male luer-lock fitting having a sleeve, the syringe not having a needle directly attached thereto; a female leer-lock fitting having a proximal end for fitting inside a male luer-lock fitting of a syringe, the syringe not having a needle directly attached thereto, the female luer-lock fitting also having a pipe integrally formed thereto, the pipe disposed within and extending out of a proximal end of the female luer-lock fitting, the pipe shaped to attach to the male luer-lock fitting so as to form a sealed flow path from the syringe through the male luer-lock fitting to the female luer-lock fitting that bypasses dead space in the male luer-lock fitting and in the female luer-lock fitting. 8C2) the cross-section of barrel 516 is normally in the shape of a circle 577 of diameter D, the circle looking like it has a piece missing (see FIG. 8C1). At the point of the bypass channel 590 below second plunger 570, the cross-section of barrel 516 may become an ordinary circle 578 of diameter is D (see FIG. 8C2). In this alternative embodiment, since the external circumference of second plunger 570 may be shaped similar to that of circle 577, when the second plunger 570 is depressed into the area of bypass channel 590, second plunger no longer seals against fluid flow.

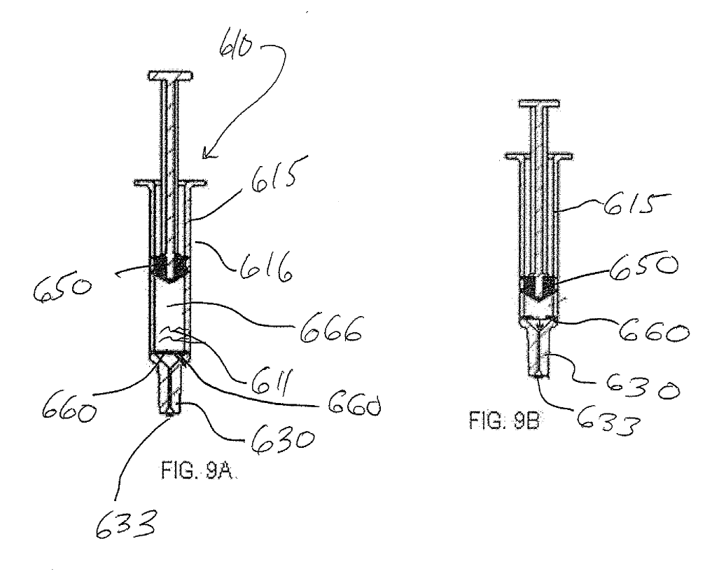

In another configuration, shown in FIG. 9A-98, for isolating fluid from microneedles or from other needles prior to injection, a pre-filled injection device 610 may include a syringe 615 having a barrel 616 pre-filled with a fluid 611 downstream of a plunger 650. As shown in FIG. 9A prior to injection, a thin membrane 660 or diaphragm 660 may be mounted between drug fluid 611 and a microneedle hub. The microneedle hub may be attached to a distal end of syringe 615. Membrane 660 may be mounted at a distal end 617 of barrel 616. This may create a sealed chamber 666 inside the pre-filled syringe 615 prior to injection. Plunger 650 may form a top boundary of the sealed chamber 666 while membrane 660 may form a lower boundary of the sealed chamber 666 within barrel 616.

As shown in FIG. 9B, when the piston 651 is depressed, plunger 650 exerts a downward force on the chamber 666 containing fluid 611. Diaphragm or membrane 660 may be designed to be breakable from the downward pressure exerted on it from the fluid 611 in the chamber 666. Accordingly, although fluid 611 may be isolated from any microneedles prior to injection, during injection fluid 611 may be able to flow to the microneedles.

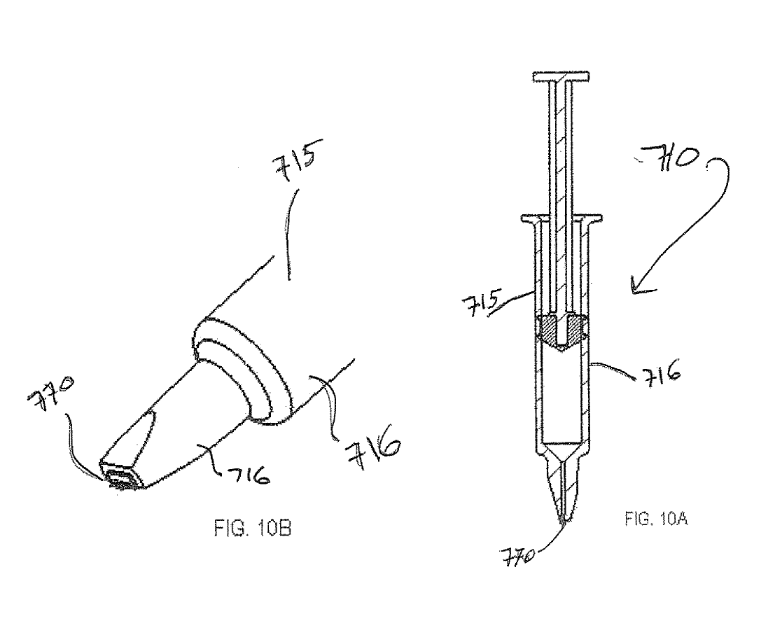

In another configuration of the present invention in which the silicon microneedle chip is mounted directly onto the syringe as shown in FIG. 10A and FIG. 10B, despite the difficulty of bonding a glass barrel of a syringe to a silicon microneedle chip, there is presented an injection device 710 comprising: a pre-filled syringe 715 including a glass barrel 716 and a microneedle chip 770. The silicon microneedle chip 770 may be bonded to the glass barrel 716 using one of several bonding processes.

Microneedle chip 770 may be attached to the glass barrel 716 containing the ready-to-use drug or compound reliably, easily, accurately and rapidly. The bonding may be sealing and be able to withstand high pressure. Preferably, the bonding materials should be made of fully biocompatible materials. In order to bond Silicon to glass structure two main physical methods may be used: bonding based on a (i) diffusion process and (ii) adhesive bonding.

Bonding may be based on a diffusion process. For example, thermal bonding occurs by diffusion and migration of atoms between two material surfaces. The surface could be silicon to silicon or glass to glass or glass to silicon etc. The thermal bonding also known as anodic bonding, electrostatic bonding, or the Malloroy process, is used for joining glass to silicon. The main utility of the process stems from the relatively low temperature process. Since the glass and silicon remain rigid during anodic bonding, it is possible to attach glass to silicon surfaces, preserving etched features in both the glass or the silicon. Further, treated silicon (for example doping with boron (B) can further facilitate these processes) The bond can be established between sodium rich glass, for example, Corning (Pyrex) and virtually any metal. Additional examples may include soda lime glass or potash soda glass or Alumino silicate glass as well. Bonding can also be accomplished between glass and silicon on a hot plate in, open atmosphere or using vacuum at temperatures between 180-500° C. The required temperature may be achieved by heating the entire device in an oven or the like, or by local heating, such as by the aforementioned anodic bonding technique, thereby inducing localized heating and diffusion bonding. Typical voltage for anodic bonding, depending on the thickness of the glass and the temperature, range from 200-1000 volts. Typical commercial instruments for anodic bonding are commercially available from Electrical Vision Co.

Adhesive bonding may also be used to bond the glass barrel to the silicon chip. Various processes and materials are known in the art for adhesive bonding of silicon to glass (as well as silicon to silicon, glass to glass etc). Examples would be UV bonding (i.e., using plastic bonding materials such as those sold under the name Henkel Loctite®) and epoxy bonding.

It should be noted that the above configurations of the present invention are non-limiting and may be combined. In addition, the present invention may also be viewed as a method of preparing a microneedle injection device, comprising using an aspiration needle affixed to a distal end of a disposable syringe to fill the syringe with a drug, the aspiration needle projecting from an aspiration needle hub; and placing a microneedle hub having a septum disposed therein onto the aspiration needle hub so that the aspiration needle pierces the septum, the septum sealing a flow path between the microneedle hub and the syringe. The microneedle injection device may be the device 310 shown in FIG. 5A, FIG. 5B and FIG. 5C.

In general, a variety of materials may be used for the making of the structures provided. For example, syringe materials may include, at a minimum, glass or polymer (including PC, PP and others), hub materials may preferably be made from polymer (including PC, PP and others) but could also be made from other materials. Sealing elements could be made from various elastomers, such as those used in the industry. Silicone derivatives or rubbers may be employed for any such component.

In addition, certain embodiments (for example the embodiment shown in FIGS. 7A-7D and the embodiment shown in FIGS. 8A-8C) may also utilize primary containers other than pre-filled and filled syringes as injection containers. Examples of other injection containers besides syringes include containers more commonly used in pen injectors (cartridges), auto injectors and pump sets, blisters or other containers.

The drugs that could be delivered may be anything that could be used in medicine, aesthetics and cosmetics. These may include liquids and in some cases non-liquid formulations or substances. Additional elements such as safety syringe concepts, safety shields, safety needles, safety vial withdrawing systems and the like could be used in combination with some of the embodiments. The actuation of the different parts in the apparatus described above may be performed manually, and in various cases may also be performed mechanically (through spring or pressure mechanisms and other mechanisms) and even electronically.

The term “microneedle hub 30” as used herein is refers to a microneedle adaptor or microneedle interface, as can be appreciated from the drawings.

While the invention has been described with respect to a limited number of embodiments, it will be appreciated that many variations, modifications and other applications of the invention may be made. Therefore, the claimed invention as recited in the claims that follow is not limited to the embodiments described herein.

1-27. (canceled)

28. A coupler that reduces dead space for fluids in an injection device, comprising: a male fitting of an injection container; a female fitting having a proximal end for fitting inside the male fitting; and an insert disposed within the proximal end of the female fitting and disposed within the male fitting for minimizing fluid remaining in the injection container and in the female fitting after injection, the insert shaped so as to maintain a flow path between the injection container and a needle attached to the female fitting.

29. The coupler of claim 28, wherein the female fitting has a distal end attached to a microneedle.

30. The coupler of claim 28, wherein the injection container is a syringe, the male fitting is a male luer-lock fitting of the syringe, the female fitting is a female luer-lock fitting having a proximal end for fitting inside the male luer-lock fitting, and the insert is disposed within and attached to the proximal end of the female luer-lock fitting and disposed within the male luer-lock fitting for minimizing fluid remaining in the syringe and in the female luer-lock fitting after injection, the insert shaped so as to maintain the flow path between the syringe and the needle attached to the female fitting.

31. The coupler of claim 30, wherein the insert has an external flow channel for maintaining the flow path.

32. A pre-filled injection device, comprising: a microneedle hub; a syringe pre-filled with a fluid and having a needle at a distal end of the syringe; an activation connector connected at a first end of the activation connector to a proximal end of the microneedle hub and connected to the distal end of the syringe at a second end of the activation connector;
a septum fitted in the microneedle hub such that in a stored position of the injection device the needle extends into the microneedle hub without piercing the entire septum and in an active position of the injection device the needle penetrates the septum entirely to maintain a flow channel between the fluid and a microneedle chip in the microneedle hub.

33. The injection device of claim 32, wherein in the active position the septum prevents the fluid from flowing back to the syringe.

34. The injection device of claim 32, wherein the activation connector and the microneedle hub have mating threads to allow rotational movement of the microneedle hub relative to the activation connector and thereby linearly move the microneedle relative to the syringe.

35. The injection device of claim 32, wherein the proximal end of the microneedle hub is fitted into the activation connector by a frictional fit that allows axial adjustment of the microneedle hub relative to the syringe.

36. A pre-filled injection device, comprising: an injection container pre-filled with a fluid, an insert having a diaphragm associated therewith, a proximal end of the insert fitted to a distal end of the injection container, the diaphragm sealing the distal end of the injection container;
a needle unit mated to a distal end of the insert so that activation of the needle unit moves a projecting element of the needle unit axially from a stored position in which the projecting element does not touch the diaphragm to an active position in which the projecting element pierces the diaphragm.

37. The injection device of claim 36, wherein the needle unit is mated to the distal end of the insert by threading and wherein activation of the needle unit is accomplished by rotation of the needle unit.

38. The injection device of claim 36, wherein activation of the needle unit occurs by pushing the needle unit toward the injection container.

39. The injection device of claim 36, wherein the syringe has a glass barrel and the needle unit has a microneedle.

40. A coupler for an injection device, comprising: a male luer-lock fitting of a syringe, the syringe not having a needle directly attached thereto; a female luer-lock fitting having a proximal end for fitting inside the male luer-lock fitting, the female luer-lock fitting also having a pipe integrally formed thereto, the pipe disposed within and extending out of a proximal end of the female luer-lock fitting, the pipe shaped to attach to the male luer-lock fitting so as to form a sealed flow path from the syringe through the male luer-lock fitting to the female luer-lock fitting that bypasses dead space in the male luer-lock fitting and in the female luer-lock fitting.

41. The coupler of claim 40, wherein the female luer-lock fitting forms part of a microneedle hub.

42. A female luer-lock fitting for a coupler of an injection device, comprising:
a male luer-lock fitting of a syringe, the syringe not having a needle directly attached thereto;
a female luer-lock fitting having a pipe integrally formed thereto, the pipe disposed within and extending out of a proximal end of the female luer-lock fitting, the pipe shaped to attach to the male luer-lock fitting so as to form a sealed flow path from the syringe through the male luer-lock fitting to the female luer-lock fitting that bypasses dead space in the male luer-lock fitting and in the female luer-lock fitting.

43. An injection device, comprising: an injection container including a barrel; a first plunger at an end of a piston and sealing the barrel from fluid flow; a second plunger downstream of the first plunger and sealing the barrel from fluid flow; and fluid separating the first plunger and the second plunger prior to injection, the barrel, downstream of the second plunger, having a bypass channel so that the second plunger no longer seals against fluid flow when the first plunger is depressed, wherein the injection container is a syringe pre-filled with the fluid and wherein a microneedle hub is attached to a distal end of the syringe.

44. A pre-filled injection device, comprising: an injection container having a barrel pre-filled with a fluid downstream of a plunger sealing a top boundary of the fluid in a chamber, the injection container also having a diaphragm at a distal end of the barrel sealing the fluid at a lower boundary; a microneedle hub attached to a distal end of the injection container;
the diaphragm breakable from pressure in the chamber when the piston is depressed for injection.

45. The injection device of claim 44, wherein the injection container is a syringe, a microneedle in the microneedle chip is made of silicon and the barrel is made of glass.

46. A method used in making an injection device, comprising: providing a syringe having a glass barrel; bonding the glass barrel to a silicon microneedle chip of a microneedle hub using a process selected from the group consisting of anodic bonding, thermal bonding and diffusion
